(19) United States
(12) Patent Application Publication
(10) Pub. No.: US 2020/0178921 A1
(43) Pub. Date: Jun. 11, 2020

(54) IMAGE DISPLAY APPARATUS, IMAGE DISPLAY METHOD, AND STORAGE MEDIUM

(71) Applicant: Konica Minolta, Inc., Tokyo (JP)

(72) Inventor: Takafumi NANJO, Tokyo (JP)

(21) Appl. No.: 16/705,637

(22) Filed: Dec. 6, 2019

(30) Foreign Application Priority Data
Dec. 7, 2018 (JP) 2018-229568

(51) Int. Cl. A61B 6/00; G06T 7/00

(52) U.S. Cl. CPC A61B 6/5235; G06T 7/0016; G06T 2207/10016; G06T 2207/10116; G06T 2207/30061; G06T 2207/20212

(57) ABSTRACT

An image display apparatus includes an image obtainer, a hardware processor, and a display. The image obtainer obtains a plurality of medical images of a same target region taken through serial radiography along a time axis. The hardware processor generates a display image based on the medical images obtained by the image obtainer. The display displays the display image generated by the hardware processor. The hardware processor combines the medical images into one composite medical image as the display image.

### CROSS-REFERENCE TO RELATED APPLICATION

The present invention claims priority under 35 U.S.C. § 119 to Japanese Patent Application No. 2018-229568, filed on Dec. 7, 2018, the entire contents of which are incorporated herein by reference.

### BACKGROUND

### 1. Technological Field

The present invention relates to an image display apparatus, an image display method, and a storage medium.

### 2. Description of the Related Art

There have been attempts to utilize, for diagnosis, a dynamic image of a region of a body to be diagnosed (hereinafter called a target region) taken with a semiconductor image sensor, such as an FPD (flat panel detector), instead of or in addition to conventional radiological (X-ray) still images taken with a film/screen or a photostimulable phosphor plate.

More specifically, by making use of high responsivity of a semiconductor image sensor in reading/deleting image data, a dynamic image of a target region is taken by continuously emitting pulsed radiation from a radiation source in sync with timing of reading/deleting of image data with the semiconductor image sensor and performing imaging multiple times per second.

For example, if the target region is the lungs or the heart, a dynamic state of the chest is imaged. With the sequence of images taken and displayed in order, a doctor can observe a sequence of chest movements with breathing, pulsation of the heart, and so forth.

There are also proposed various technologies using a dynamic image of the chest to extract feature quantity information useful for determining ventilation volume by breathing. For example, there is known a technology of: taking dynamic images of the chest from the front and from the side; and obtaining change volume of the lungs as ventilation volume of the lungs on the basis of change in the position of the base of the lungs and the thickness of the lungs.

By the way, the lungs damaged by the chronic obstructive pulmonary disease (COPD), which is a disease of the lungs, are irreversible, and the damaged lung parenchyma is incurable. Patients of the COPD do home oxygen therapy and/or train their respiratory muscles through respiratory organs rehabilitation in order to improve quality of their daily living. It is necessary to evaluate whether or not respiratory functions are recovered through such therapy and training of respiratory muscles. To evaluate the degree of recovery of respiratory functions, it is important to observe movement of the diaphragm, which is one of the main respiratory muscles.

In order to observe movement of the diaphragm, for example, there is proposed in JP 2012-115582A, a technology of: taking a dynamic image of the chest; calculating positions of the diaphragm on the basis of the taken dynamic image; and obtaining diagnosis aiding information on the ventilation functions, including the calculation results.

### SUMMARY

In order to check the state of a target region, such as the diaphragm, it is preferable that the shape, the position, and so forth of the target region be observed visually on the taken images.

With respect to a region that changes over time, such as the diaphragm, the shape and so forth of the region can be easily checked visually so long as the number of taken images is one.

However, one time of dynamic imaging usually generates dozens or hundreds of images, which requires much time just to check the change. Furthermore, because a doctor views the images as an animation, the doctor finds it difficult to check the change by one time of viewing, and may overlook the change. Because of such difficulty in visually checking movement of the target region, the doctor consumes much time to view the images for making diagnosis.

The present invention has been conceived in view of the above problems. The objects of the present invention include providing an image display apparatus, an image display method, and a storage medium storing an image display program that allow users to check results of dynamic imaging swiftly and improve diagnosis efficiency.

In order to achieve at least one of the abovementioned objects, according to a first aspect of the present invention, there is provided an image display apparatus including: an image obtainer that obtains a plurality of medical images of a same target region taken through serial radiography along a time axis; a hardware processor that generates a display image on the basis of the medical images obtained by the image obtainer; and a display that displays the display image generated by the hardware processor, wherein the hardware processor combines the medical images into one composite medical image as the display image.

According to a second aspect of the present invention, there is provided an image display apparatus including: an image obtainer that obtains a plurality of medical images of a same target region taken through serial radiography along a time axis; a hardware processor that extracts feature points from the respective medical images obtained by the image obtainer, and lays the extracted feature points on one selected medical image that is selected from among the medical images, thereby generating a display image; and a display that displays the display image generated by the hardware processor.

According to a third aspect of the present invention, there is provided an image display method including: obtaining a plurality of medical images of a same target region taken through serial radiography along a time axis; generating a display image on the basis of the obtained medical images; and displaying the generated display image, wherein in generating the display image, the medical images are combined into one composite medical image as the medical image.

According to a forth aspect of the present invention, there is provided an image display method including: obtaining a plurality of medical images of a same target region taken through serial radiography along a time axis; extracting feature points from the respective obtained medical images; laying the extracted feature points on one selected medical image that is selected from among the medical images, thereby generating a display image; and displaying the generated display image.

According to a fifth aspect of the present invention, there is provided a non-transitory computer-readable storage medium storing an image display program to cause a computer of an image display apparatus to: obtain a plurality of medical images of a same target region taken through serial radiography along a time axis; generate a display image on the basis of the obtained medical images; and display the generated display image, wherein the display image is one composite medical image into which the medical images are combined.

According to a sixth aspect of the present invention, there is provided a non-transitory computer-readable storage medium storing an image display program to cause a computer of an image display apparatus to: obtain medical images of a same target region taken through serial radiography along a time axis; extract feature points from the respective obtained medical images; lay the extracted feature points on one selected medical image that is selected from among the medical images, thereby generating a display image; and display the generated display image.

### BRIEF DESCRIPTION OF THE DRAWINGS

The advantages and features provided by one or more embodiments of the present invention will become more fully understood from the detailed description given hereinbelow and the appended drawings which are given by way of illustration only, and thus are not intended as a definition of the limits of the present invention, wherein:

FIG. 1 is a block diagram of main components of a medical imaging system including an image display apparatus according to an embodiment(s);

FIG. 2 is a block diagram of main components of the image display apparatus according to the embodiment(s);

FIG. 3 shows an example of a display screen of a display;

FIG. 4 shows a screen displayed on the display screen of the display when a point in the vicinity of diaphragm lines is selected;

FIG. 5 shows a screen displayed on the display screen of the display when a display image is switched to a medical image having a diaphragm line selected by a user;

FIG. 6 is a flowchart showing a process performed by the image display apparatus according to the embodiment(s); and

FIG. 7 is a flowchart showing a process performed by the image display apparatus according to the embodiment(s).

### DETAILED DESCRIPTION OF EMBODIMENTS

Hereinafter, one or more embodiments of an image display apparatus (image display system) according to the present invention will be described with reference to the drawings.

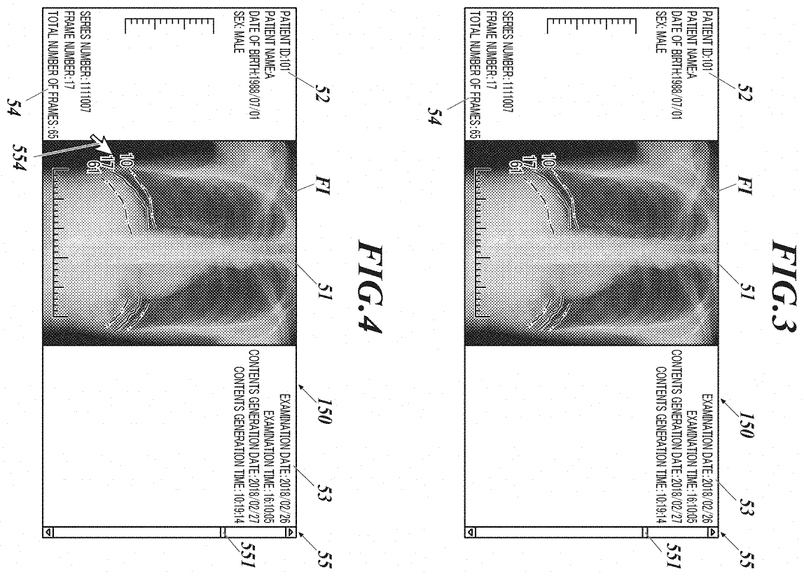

The following is a case where one apparatus (image display apparatus) includes: a controller 11 that analyzes images, controls displaying, and determines a range of medical images to be displayed; a storage 12; and a display 15 (all shown in FIG. 2), which are described below. The image display apparatus described below can be configured as an image display system. For example, it is possible to configure some or all of parts/components including a functional part analyzing images, a functional part determining a range of medical images to be displayed, a storage, and a display as individual apparatuses, and connect them as a system (i.e. image display system, not illustrated) on a network.

Although the embodiments described below have various limitations that are technically preferable to embody the present invention, the scope of the present invention is not limited to the embodiments or the illustrated examples.

### [Role of Image Display Apparatus (Image Display System)]

An image display apparatus in this embodiment is included in a medical imaging system, and obtains medical images, such as medical images constituting a radiological dynamic image (hereinafter called a dynamic image), and displays the obtained medical images including the medical images constituting the dynamic image and various kinds of information.

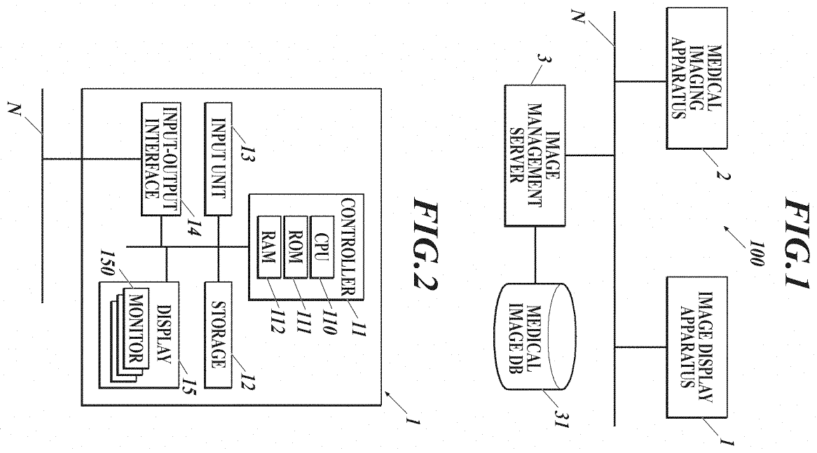

First of all, as a premise, an assumed relationship between the medical imaging system and the image display apparatus in this embodiment is described with reference to FIG. 1.

### [Configuration of Medical Imaging System]

FIG. 1 is an example of configuration of a medical imaging system 100. The medical imaging system 100 is a system installed in a medical facility, such as a hospital.

As shown in FIG. 1, the medical imaging system 100 includes an image display apparatus 1 (medical image display apparatus), a medical imaging apparatus 2, and an image management server 3. These apparatuses connect to each other for data exchange via a communication network N consisting of a communication line(s), such as a local area network (LAN) and a wide area network (WAN). Each of the components constituting the medical imaging system 100 conforms to digital imaging and communications in medicine (DICOM) standard, and communication between the components is performed in accordance with the DICOM standard.

As to each of the apparatuses constituting the medical imaging system 100, the number of apparatuses is not particularly limited. For example, as the medical imaging apparatus 2, various types of modalities, such as a computed tomography (CT) apparatus, a magnetic resonance imaging (MRI) apparatus, and a computed radiography (CR) apparatus, may be installed. The modalities to be prepared may be the same or different from one another in type. The number of modalities, combinations of the types of modalities, and so forth are determined appropriately to meet the needs of the facility, for example.

The image management server 3 is a computer that accumulates, stores, and manages image data of medical images generated by various modalities (medical imaging apparatus 2) and accessory information on the medical images. Examples of medical images include: tomograms generated by CT apparatuses and MRI apparatuses; and plain roentgenograms generated by CR apparatuses. Medical images in this embodiment include medical images constituting a dynamic image that is taken through dynamic imaging by the medical imaging apparatus 2.

Herein, dynamic imaging means imaging of a dynamic state of a target region through serial radiography along a time axis. More specifically, dynamic imaging is performed by: repeatedly emitting pulsed radiation from a radiation source (not illustrated) at predetermined time intervals (pulse emission); or continuously and seamlessly emitting radiation at a low dose rate (continuous emission), thereby continuously imaging the dynamic state of the target region multiple times so as to be an animation. A dynamic image means a sequence of images taken through such imaging and showing a dynamic state of a target region.

A series is, for example, a sequence of medical images that relate to one another, such as a group of medical images taken in a single examination for a target region of a patient. With respect to a dynamic image taken through dynamic imaging, a series is a sequence of medical images taken through dynamic imaging, which is performed by continuously imaging the target region.

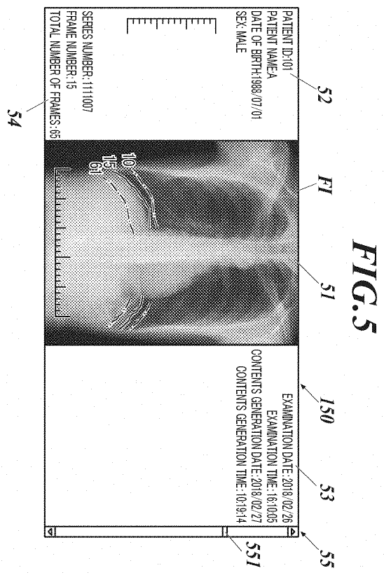

Medical images constituting a dynamic image are called frame images (frame image FI in FIG. 3 and so forth). The frame images constituting one series have their respective frame numbers in the imaging order.

In this embodiment, dynamic imaging is performed for a target region having cyclical movement, such as the lung fields, the diaphragm, and the heart. However, these are not the limitation of the target region of dynamic imaging.

The image management server 3 includes a medical image database (DB) 31 that consists mainly of a hard disk. The medical image DB 31 stores data of medical images and accessory information on the medical images.

The medical image DB 31 stores medical images in the DICOM file format conforming to the DICOM standard. A DICOM file includes an image part and a header part. In the image part, actual data of medical images is written, and in the header part, accessory information on the medical images is written.

The accessory information includes, for example, patient information, examination information, series information, and image detailed information.

The patient information includes various kinds of information on a patient of medical images, such as patient identification information for identifying the patient (e.g. patient ID), patient name, sex, and date of birth.

The examination information includes various kinds of information on an examination, such as examination identification information for identifying the examination (e.g. examination ID), the examination date, and a doctor in charge.

The image detailed information includes various kinds of information on medical images, such as the time when the images were generated, the name of a file path indicating the location where the medical images are stored, comments on the examination, the location where a lesion is found, and the found result.

The series information includes various kinds of information on a series of medical images, such as: a series number for identifying the series in an examination; type of modality (medical imaging apparatus 2) that generated the medical images of the series; examined region; and, if the medical images constitute a dynamic image, the total number of the frame images in the series and frame numbers, which are given to the respective frame images in the series.

As described above, the frame numbers 1 to n (n: the total number of frame images FI) indicate which number in the imaging order the respective frame images generated as one series (the frame images FI of the dynamic image) have been taken.

The image management server 3 reads out, in response to a request for medical images sent by the image display apparatus 1 via the communication network N, medical image data from the medical image DB 31 and sends the read data to the image display apparatus 1.

### [Configuration of Image Display Apparatus (Image Display System)]

Configuration of the image display apparatus 1 (or the image display system; the same applies to the following) in this embodiment is described.

FIG. 2 is a block diagram showing configuration of the image display apparatus 1 according to this embodiment.

In this embodiment, the image display apparatus 1 is a computer that includes a controller 11 (hardware processor), a storage 12, an input unit 13, an input-output interface 14, and a display 15 that connect to a bus as shown in FIG. 2.

The image display apparatus 1 is connected to the communication network N via the input-output interface 14.

In this embodiment, the input-output interface 14 is an image obtainer that obtains, via the communication network N, medical images taken through radiography.

The input-output interface 14 as the image obtainer obtains, from the image management server 3 (the medical image DB 31 of the image management server 3), a dynamic image of a dynamic state of a target region taken through serial radiography along a time axis.

The image display apparatus 1 may be a general-purpose computer, or can be an apparatus dedicated for medical images.

The image display apparatus 1 may be an integrated apparatus (not illustrated) that includes: a control device, such as a console, controlling an imaging operation of the medical imaging apparatus 2; and an image saving database for saving medical images, such as taken frame images FI.

The image display apparatus 1 may be: a desktop personal computer; a notebook computer; a tablet-type terminal device; or a portable terminal device, such as a smartphone.

The controller 11 includes a central processing unit (CPU) 110, a read only memory (ROM) 111, and a random access memory (RAM) 112.

The ROM 111 stores various programs that are performed by the CPU 110, such as a system program and a display control process program (image display program) for displaying images, and data required for performing these programs. These programs are stored in the ROM 111 in a form of computer readable program code. The CPU 110 operates by following the program code.

The RAM 112 forms, in various kinds of processing performed and controlled by the CPU 110, a work area to temporarily store various programs read out from the ROM 111, input/output data, parameters, and so forth.

In this embodiment, the controller 11 controls the display 15 (monitor 150), which is described below, displaying medical images.

The controller 11 in this embodiment also determines whether or not medical images to be displayed on the display 15 (monitor 150) include medical images constituting a dynamic image of a target region taken through serial radiography along a time axis.

As an example of methods used by the controller 11 for determining whether or not medical images to be displayed on the display 15 include medical images constituting a dynamic image, the controller 11 refers to accessory information on the medical images in an examination specified by a user. When determining, on the basis of the accessory information on the medical images, that a predetermined number of images or more has been taken continuously, the controller 11 determines the group of images to be a dynamic image. The predetermined number of images in this case is set to an appropriate number, for example, 50 images (or more).

The method used by the controller 11 for determining whether or not medical images to be displayed on the display 15 include medical images constituting a dynamic image is not limited to this, and may be any other methods. For example, if the header part of a DICOM file includes, as accessory information, identification information or the like indicating that the medical images constitute a dynamic image, the controller 11 determines that the group of medical images having such accessory information to be the dynamic image.

The controller 11 also extracts feature points from the respective medical images. In this embodiment, the controller 11 extracts, as feature points, diaphragm lines indicating positions of the diaphragm in the respective medical images.

More specifically, the controller 11 detects the positions of the diaphragm in the respective frame images FI constituting the dynamic image by analyzing the frame images FI. The controller 11 then stores, in the storage 12 or the like, the detection results of the diaphragm lines indicating the positions of the diaphragm as accessory information on the respective frame images FI.

The controller 11 in this embodiment determines cycles of movement of the target region by analyzing the frame images FI constituting the dynamic image.

For example, if the target region having cyclical movement is the human lung fields and the lung fields are imaged from the front, and a period of one respiration in the lung fields is treated as one cycle, the controller 11: analyzes the frame images FI appropriately; detects at least the highest and lowest positions of the diaphragm moving up and down with breathing; and identifies the medical images indicating the highest and lowest positions of the diaphragm. Thus, the controller 11 identifies the medical images indicating the start and the end of one cycle of the movement of the target region.

More specifically, the controller 11 detects the positions of the diaphragm in the respective frame images by analyzing the frame images. The image in which the diaphragm locates at the highest position indicates a state when an exhalation period ends, namely when air is completely exhaled. From this point, an inhalation period starts and the diaphragm gradually moves down as air is inhaled. The image in which the diaphragm locates at the lowest position indicates a state when the inhalation period ends, namely when air is inhaled to the limit. The controller 11 determines that a sequence of images starting from the image in which the diaphragm locates at the highest position to the image in which the diaphragm locates at the lowest position are images corresponding to the inhalation period. Contrariwise, the controller 11 determines that a sequence of images starting from the image in which the diaphragm locates at the lowest position to the image in which the diaphragm locates at the highest position are images corresponding to the exhalation period. The controller 11 also determines that a set of the images corresponding to the inhalation period and the images corresponding to the exhalation period being sequential are images corresponding to one respiration period.

One respiration period in this case may start from an inhalation period followed by an exhalation period, or from an exhalation period followed by an inhalation period, thereby being a set of an inhalation period and an exhalation period.

There are not specific limitations to the methods used by the controller 11 for: determining cycles of movement of the target region; and identifying the images indicating the start and the end of one cycle of the movement of the target region, and various methods can be adopted, for example, methods disclosed in WO 2009/090894, JP 2009-273671, and JP 2009-153678.

Furthermore, the controller 11 in this embodiment generates a display image that is displayed on the display 15 (monitor 150) on the basis of medical images obtained by the input-output interface 14 as the image obtainer.

There is not particular limitation to a range of medical images among the obtained medical images on the basis of which the controller 11 generates the display image. For example, the controller 11 may generate the display image on the basis of all the obtained medical images, or on the basis of a series, which is a sequence of medical images, as one unit. If the controller 11 determines cycles of movement of the target region as described above, the controller 11 may generate the display image on the basis of medical images corresponding to one cycle (e.g. one human respiration period consisting of an inhalation period and an exhalation period or half a human respiration period consisting of an inhalation period or an exhalation period) as one unit.

In the embodiment described below, among the obtained medical images, with respect to medical images determined to be a dynamic image, the controller 11 generates the display image on the basis of medical images corresponding to one human respiration period, which consists of an inhalation period and an exhalation period, as one unit.

The controller 11 in this embodiment lays feature points (diaphragm lines in this embodiment) extracted from the medical images as one unit on one medical image selected from among the medical images as one unit, thereby generating the display image.

In this embodiment, the selected medical image is a medical image selected by a user from among the medical images that may be displayed on the display 15.

The method of selecting the medical image is not particularly limited. For example, if medical images corresponding to one human respiration period, which consists of an inhalation period and an exhalation period, are treated as one unit as described above, any of the following images may be selected automatically: an image taken at the timing when an inhalation period ends and an exhalation period starts; an image taken at the timing when an exhalation period ends and an inhalation period starts; and an image taken at around either of the above timings.

In this embodiment, the controller 11 identifies: a diaphragm line indicating the position of the diaphragm in the selected medical image; and at least a diagram line indicating the highest position of the diaphragm (i.e. the position of the diaphragm when the exhalation period ends, namely when air is completely exhaled), which moves up and down with breathing, and a diaphragm line indicating the lowest position of the diaphragm (i.e. the position of the diaphragm when the inhalation period ends, namely when air is inhaled to the limit). The controller 11 then lays, on the selected medical image, the diaphragm lines indicating at least the highest position of the diaphragm, the lowest position of the diaphragm, and the position of the diaphragm in the selected medical image, thereby generating a feature-point-laid medical image as the display image as shown in FIG. 3.

In FIG. 3, the 17th frame image FI in one series consisting of 65 frame images in total is selected. In the selected medical image, the diaphragm line indicating the position of the diaphragm in the selected medical image is represented by a solid line; and the diagram lines indicating the highest and lowest positions of the diaphragm are represented by broken lines.

Near the respective diaphragm lines, frame numbers (i.e. information on the feature points) indicating the frame images FI to which the respective diaphragm lines belong are displayed along with the diaphragm lines.

The feature points (diaphragm lines in this embodiment) may be distinguished by types of lines as shown in FIG. 3, or by colors of lines if the display 15 can perform full-color display. If the display image includes information on the feature points, such as text information exemplified by the frame numbers, it is possible to distinguish the feature points on the basis of the displayed text information without changing types or colors of the lines.

It is not necessary to display the text information exemplified by the frame numbers (i.e. the information on the feature points), and only the feature points (diaphragm lines in this embodiment) may be displayed without the text information.

Furthermore, the controller 11 does not have to generate the display image by laying, on the selected medical image, three diaphragm lines indicating the highest and lowest positions of the diaphragm and the position of the diaphragm in the selected medical image.

For example, the controller 11 may lay all the feature points (diaphragm lines in this embodiment) extracted by the controller 11 from the medical images treated as one unit on the medical image selected from among the medical images, thereby generating the feature-point-laid medical image as the display image.

Furthermore, the controller 11 may generate the display image on the basis of processed medical images that are the medical images obtained by the input-output interface 14 as the image obtainer and processed.

Examples of the processed medical images are: images in which movement/shifts of the body of a patient has/have been corrected (registration images) if the patient moved or the body of the patient shifted over time during imaging; and images in each of which the diaphragm has been emphasized for easy understanding of the movement of the diaphragm (emphasis images).

Such image processing may be performed by the controller 11 or other functional parts.

In this embodiment, the monitor 150 of the display 15 is combined with a touchscreen as the input unit 13 as described below. When a user (e.g. doctor) performs an input operation on the display image displayed on the monitor 150 of the display 15 (i.e. operation of touching/tapping the display image on the monitor 150 combined with the touchscreen), the input unit 13 detects and receives the input operation and sends, to the controller 11, a signal corresponding to the input operation.

When receiving the signal from the input unit 13, the controller 11 identifies the location of the input operation detected and received by the input unit 13, and identifies the medical image having the diaphragm line that is closest to the location of the input operation.

The storage 12 includes, for example, a nonvolatile semiconductor memory and/or a hard disk drive (HDD), and stores such as various data.

The storage 12 stores, for example, medical images obtained by the input-output interface 14 as the image obtainer.

The input unit 13 includes, for example, a keyboard, a mouse, and a touchscreen. As described above, the touchscreen is formed over the monitor 150 of the display 15 as one body.

In this embodiment, the input unit 13 detects and receives the input operation input by the user touching/tapping the image displayed on the monitor 150. When receiving the input operation, the input unit 13 outputs, to the controller 11, a signal corresponding to the input operation.

The input operation received by the input unit 13 is not limited to the touching/tapping of the touchscreen. The input operation may be such as clicks on the monitor 150 with the mouse. FIG. 4 shows, as an example, the input operation of clicking an image on the monitor 150 with a pointer 554 indicating the operation position of the mouse.

In this case, the controller 11 identifies where on the display screen the pointer 554 points, namely identifies the coordinates on the display screen where the input operation is performed.

The display 15 displays medical images and various kinds of information on the medical images.

In this embodiment, the display 15 displays a display image generated by the controller 11.

In this embodiment, the display 15 performs a display process in accordance with the control of the controller 11.

In this embodiment, the display image (feature-point-laid medical image) includes feature points (diaphragm lines) and information on the feature points (i.e. text information, such as frame numbers). The display 15 displays the display image including the feature points (diaphragm lines) and the information on the feature points by being laid on the display image.

The display 15 includes the monitor 150 that consists of, for example, a cathode ray tube (CRT) and/or a liquid crystal display (LCD).

In this embodiment, the display 15 includes a plurality of monitors 150. At least one of the monitors 150 is a high-definition monitor for the user to make image interpretation.

As an example of detailed configuration of the display 15, the display 15 includes: a normal monitor 150 for displaying a screen of a list of such as patient information and examination information for the user to select medical images to interpret; and a high-definition monitor 150 for the user to interpret the medical images. As to each of the normal monitor 150 and the high-definition monitor 150, the number of monitors may be one or more.

In this embodiment, at least the high-definition monitor 150 for image interpretation is combined with the touchscreen, which is the input unit 13. When the user performs the input operation by, for example, touching/tapping the display screen of the monitor 150 with a finger or a touch pen, a predetermined input instruction signal is generated in accordance with the location of the input operation (e.g. touching operation) and sent to the controller 11.

FIG. 3 shows an example of the monitor 150 in this embodiment. The example in FIG. 3 is the high-definition monitor 150, which displays such as medical images, in particular medical images constituting a dynamic image that may be taken in an examination selected by the user, so that the user interprets the dynamic image.

As shown in FIG. 3, at the near center of the display screen of the monitor 150 for image interpretation, an image display region 51 for displaying a frame image FI is provided.

The left and right sides of the image display region 51 include: a patient information display region 52 for displaying patient information; an examination information display region 53 for displaying examination information on the frame image FI displayed in the image display region 51; and an image information display region 54 for displaying (i) information on the frame image FI displayed in the image display region 51 and (ii) information on a series that includes the frame image FI.

In FIG. 3, the upper left of the display screen is allocated for the patient information display region 52 for displaying patient information, such as patient ID, patient name, date of birth, and sex. The upper right of the display screen is allocated for the examination information display region 53 for displaying examination information, such as: an examination date and time; and a date and time when contents including the frame image FI were generated. The lower left of the display screen is allocated for the image information display region 54 for displaying such as a frame number for the frame image FI, the total number of the frame images, and a series number unique to the series that includes the frame image FI.

The display regions that are set in the display screen of the monitor 150 are not limited to those exemplified here. The items displayed in each of the display regions 51 to 54 are not also limited to those exemplified here. The arrangement of the display regions 51 to 54 is not also limited to the example illustrated.

The types and the arrangement of the display regions 51 to 54, the items displayed in each of the display regions 51 to 54, and so forth may be changed and customized appropriately by the user.

Furthermore, on a side or the like of the display screen of the monitor 150 (in this embodiment, the right edge as shown in FIG. 3 and so forth), a scrollbar 55 is arranged so as to extend along the up-and-down direction of the display screen of the monitor 150.

The scrollbar 55 has, for example, a scroller 551 for frame images FI that can be displayed in the image display region 51. When the user scrolls the scrollbar 55 (i.e. moves the scroller 551 on the scroll bar 55), the image displayed in the image display region 51 is switched to a frame image FI corresponding to the location of the scroller 551 in accordance with the scrolling operation. That is, locations of the scroller 551 correspond to the respective frame images FI that are included in one series and can be displayed in the image display region 51.

The scrollbar 55 is not essential, and characteristics of the scrollbar 55 including the location and the components thereof are not limited to those exemplified here.

The controller 11 identifies a medical image having a diaphragm line that is closest to the location of the input operation detected and received by the input unit 13, and causes the display 15 to display the identified medical image on the monitor 150 by replacing the display image currently displayed in the image display region 51 with the identified medical image.

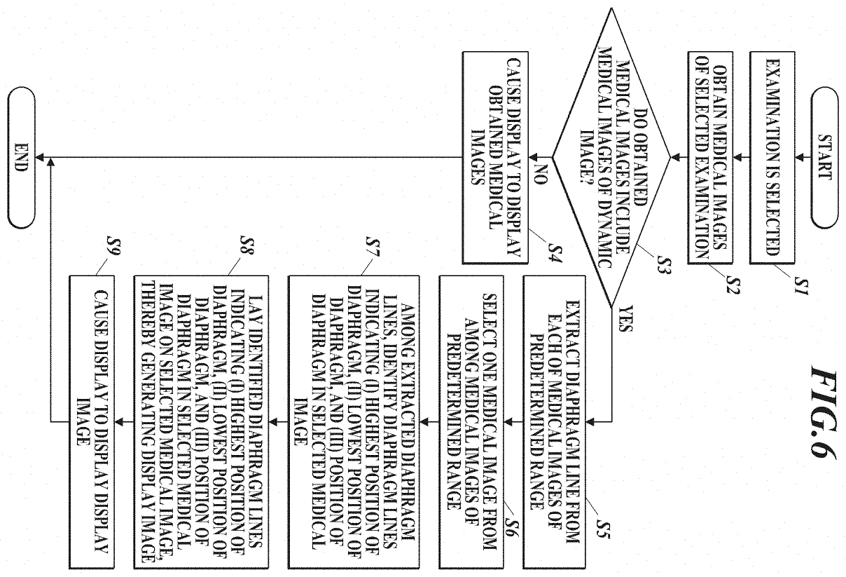

For example, when the user performs the input operation (e.g. clicks a point in the vicinity of the diaphragm lines with the pointer 554 of the mouse) on the display image as shown in FIG. 4, the controller 11 identifies the medical image having the diaphragm line that is closest to the location of the input operation. If the identified medical image is the frame image with the frame number 15, the display image currently displayed (in FIG. 4, the frame image FI with the frame number 17) is switched to the frame image FI with the frame number 15. Thus, the frame image FI with the frame number 15 is displayed in the image display region 51 of the monitor 150, as shown in FIG. 5.

### [Function of Image Display Apparatus and Image Display Method]

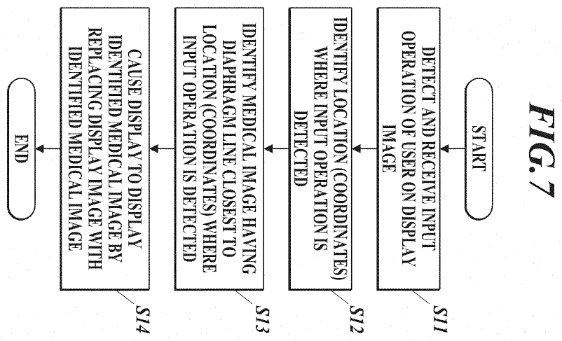

Next, an image display method in this embodiment is described with reference to FIG. 6 and FIG. 7.

When a user interprets images, the image display apparatus 1 firstly causes the display 15 to display a screen of a list of examinations or the like.

When the user specifies, on the display screen, such as a desired examination about which the user intends to make image interpretation, the controller 11 selects the examination (Step S1 in FIG. 6).

When the controller 11 selects the examination, the image display apparatus 1 sends an image obtainment request to the image management server 3. The image management server 3 retrieves, from the medical image DB 31, image data of the requested examination, and sends the data to the image display apparatus 1 via the communication network N.

The image display apparatus 1 obtains the data of the medical images and the accessory information appended to the medical images via the input-output interface 14, which is the image obtainer (Step S2).

When obtaining the medical images, the controller 11 determines whether or not the obtained medical images include medical images of a dynamic image (Step S3). If the controller 11 determines that the obtained medical images do not include medical images of a dynamic image (Step S3: NO), the controller 11 causes the display 15 to display, on the monitor 150 (the high-definition monitor for image interpretation), the obtained medical images as they are (Step S4).

If the controller 11 determines that the obtained medical images include medical images of a dynamic image (Step S3: YES), the controller 11 extracts a diaphragm line as a feature point from each of medical images of a predetermined range (Step S5).

Here, as described above, a predetermined range of medical images is: one series of medical images; or medical images corresponding to one respiration period among medical images constituting one series. The predetermined range of medical images may be set beforehand, or may be set appropriately by the user.

The controller 11 then selects one medical image from the medical images of the predetermined range in accordance with, for example, the specification by the user (Step S6).

In this embodiment, the controller 11 identifies, among the diaphragm lines extracted in Step S5, three diaphragm lines indicating (i) the position of the diaphragm in the medical image selected in Step S6, (ii) the highest position of the diaphragm, and (iii) the lowest position of the diaphragm (Step S7). The controller 11 then lays the identified three diaphragm lines on the medical image selected in Step S6, thereby generating a display image (Step S8).

The controller 11 then causes the display 15 to display the generated display image on the monitor 150 (high-definition monitor for image interpretation) (Step S9).

As shown in FIG. 7, when the input unit 13 detects and receives an input operation input by the user to the display image displayed on the monitor 150 of the display 15 (Step S11), the controller 11 identifies the location (coordinates) where the input operation is detected (Step S12).

The controller 11 then identifies the medical image having the diaphragm line that is closest to the location (coordinates) (Step S13), and causes the display 15 to display the identified medical image on the monitor 150 by replacing the display image with the identified medical image (Step S14).

As described above, the image display apparatus 1 according to this embodiment: obtains a plurality of medical images of the same target region taken through serial radiography along a time axis; extracts feature points from the respective obtained medical images; lays the extracted feature points on one selected medical image that is selected from among the medical images, thereby generating a display image; and displays the display image on the display (monitor 150 of the display 15).

This allows the selected medical image to include important information for image interpretation and diagnosis. Even if there is a large number of medical images taken in an examination about which image interpretation and diagnosis should be made, a user can make appropriate image interpretation and diagnosis swiftly and efficiently by viewing the selected medical image.

Furthermore, in this embodiment, the image display apparatus 1: extracts diaphragm lines as feature points indicating positions of the diaphragm, which moves up and down with breathing, from the respective medical images; and lays, on the selected medical image, at least among the diaphragm lines (i) a diaphragm line indicating the highest position of the diaphragm, (ii) a diaphragm line indicating the lowest position of the diaphragm, and (iii) a diaphragm line indicating the position of the diaphragm in the selected medical image, thereby generating a feature-point-laid medical image as the display image.

In order to learn the state of activities of the diaphragm and the state of recovery from a disease, it is particularly important to grasp the highest and lowest positions of the diaphragm between which the diaphragm moves.

In this embodiment, the diaphragm lines indicating (i) the highest position of the diaphragm, (ii) the lowest position of the diaphragm, and (iii) the position of the diaphragm in the selected medical image are displayed together on the display image.

By viewing one display image, the user can check the state of the diaphragm easily and make appropriate image interpretation and diagnosis swiftly and efficiently.

In this embodiment, the display image includes information on the feature points as well as the feature points themselves, and the display 15 displays the display image including the information on the future points. This makes the display image more comprehensible, and by viewing the display image, the user can make appropriate image interpretation and diagnosis more efficiently.

Furthermore, in this embodiment, the input unit 13 detects and receives an input operation on the display image displayed by the display 15. The controller 11 identifies the location of the input operation, and identifies a medical image having a diaphragm line closest to the location of the input operation among the diaphragm lines. By replacing the display image with the identified medical image, the controller 11 causes the display (monitor 150 of the display 15) to display the medical image having the diaphragm line closest to the location of the input operation.

Thus, the medical image that the user wants to view can be displayed easily and swiftly, which is user-friendly.

The controller 11 may generate the display image on the basis of, among the obtained medical images, a series being a sequence of medical images as one unit. This allows the user to interpret images on a series basis and make swift diagnosis.

If the target region is the human lung fields including the diaphragm, the controller 11 may generate the display image on the basis of, among the obtained medical images, medical images corresponding to at least one human respiration period consisting of an inhalation period and an exhalation period as one unit. This allows the user to observe movement of the lung fields on a respiration basis and make detailed image interpretation.

Thus, the user can easily and swiftly interpret images about the state of activities of the lung fields including the diaphragm, and accurately judge: whether or not hypofunction of the lungs is observed; how the state of the lung fields is after a treatment; and so forth.

If the controller 11 determines that the obtained medical images include medical images of a dynamic image of the target region that has cyclical movement, the dynamic image being taken through serial radiography along a time axis, the controller 11 may generate the display image on the basis of the dynamic image. This allows the user to easily and swiftly interpret the dynamic image, which often consists of a large number of images, and make diagnosis.

The controller 11 may generate the display image on the basis of processed medical images that are the obtained medical images subjected to various kinds of image processing. This allows the user to make appropriate image interpretation and diagnosis on the basis of the display image easier to view and comprehend.

### [Modification]

Naturally, the present invention is not limited to the above embodiment and can be modified appropriately within the scope of the present invention.

For example, in the above embodiment, the image management server 3 includes the medical image DB 31, which consists mainly of a HDD and stores data of medical images and accessory information on the medical images. However, the configuration of the image management server and the medical image DB is not limited to the configuration exemplified here. The image management server and the medical image DB may be configured in other ways so long as they store and manage medical images taken by the medical imaging apparatus 2.

For example, with the recent spread of cloud servers, it is possible that all of data is stored and managed in a cloud data center located outside medical facilities, and the medical facilities keep latest data only. In such a case, the image display apparatus 1 that is in a medical facility and requests medical images as a request sender in accordance with operations of a user may: directly access a storage server (cloud server, not illustrated) that stores and manages images; select medical images as a target of medical cooperation; and obtain data of the medical images.

In the above embodiment, the controller 11 extracts feature points from medical images and lays the extracted feature points on one medical image selected from among the medical images, thereby generating a display image. However, the display image generated by the controller 11 is not limited to this.

For example, the controller 11 may combine medical images into one composite medical image as the display image. It is not necessary to combine all the taken medical images. Only some of the medical images may be combined into the composite medical image.

Furthermore, the controller 11 may lay feature points extracted from the respective medical images on the composite medical image, into which the medical images are combined, thereby generating a feature-point-laid medical image as the display image.

As described above, if the controller 11 generates a display image on the basis of a plurality of medical images of the same target region taken through serial radiography along the time axis to cause the display 15 to display the display image on the monitor 150, the controller 11 may combine the medical images into one composite medical image as the display image. Thus, even if there is a large number of medical images taken in an examination about which image interpretation and diagnosis should be made, the user can interpret the medical images by viewing one composite medical image as the display image, into which the medical images are combined.

This allows the user to make appropriate image interpretation and diagnosis swiftly and efficiently.

Furthermore, in this modification, the controller 11 extracts feature points from the respective medical images and lays the extracted feature points on the composite medical image, thereby generating a feature-point-laid medical image as the display image. This allows the user to make appropriate image interpretation and diagnosis more swiftly and efficiently.

In the above embodiment, the controller 11 extracts diaphragm lines indicating positions of the diaphragm as an example of feature points, but the feature points extracted by the controller 11 are not limited to the diaphragm lines.

For example, the feature points may relate to the heart (e.g. shape of the heart), which repeats expansion and contraction.

Although some embodiments of the present invention have been described and illustrated in detail, the disclosed embodiments are made for purposes of illustration and example only and not limitation. The scope of the present invention includes the scope of the present invention described in the scope of claims and the scope of their equivalents.

What is claimed is:

1. An image display apparatus comprising:
an image obtainer that obtains a plurality of medical images of a same target region taken through serial radiography along a time axis;
a hardware processor that generates a display image based on the medical images obtained by the image obtainer; and
a display that displays the display image generated by the hardware processor, wherein
the hardware processor combines the medical images into one composite medical image as the display image.

2. The image display apparatus according to claim 1, wherein the hardware processor
extracts feature points from the respective medical images, and
lays the extracted feature points on the composite medical image, thereby generating a feature-point-laid medical image as the display image.

3. An image display apparatus comprising:
an image obtainer that obtains a plurality of medical images of a same target region taken through serial radiography along a time axis;
a hardware processor that
extracts feature points from the respective medical images obtained by the image obtainer, and
lays the extracted feature points on one selected medical image that is selected from among the medical images, thereby generating a display image; and
a display that displays the display image generated by the hardware processor.

4. The image display apparatus according to claim 3, wherein
the feature points extracted by the hardware processor are diaphragm lines indicating positions of a diaphragm that moves up and down with breathing, and
the hardware processor lays, on the selected medical image, at least among the diaphragm lines (i) a diaphragm line indicating a highest position of the diaphragm, (ii) a diaphragm line indicating a lowest position of the diaphragm, and (iii) a diaphragm line indicating a position of the diaphragm in the selected medical image, thereby generating the display image.

5. The image display apparatus according to claim 3, wherein
the display image includes information on the feature points as well as the feature points, and
the display displays the display image including the information on the feature points.

6. The image display apparatus according to claim 4, further comprising an input unit that detects and receives an input operation on the display image displayed by the display, wherein
the hardware processor identifies a location of the input operation detected and received by the input unit, and identifies a medical image having a diaphragm line closest to the location of the input operation among the diaphragm lines, and
by replacing the display image with the identified medical image, the display displays the medical image identified by the hardware processor.

7. The image display apparatus according to claim 1, wherein the hardware processor generates the display image based on, among the medical images obtained by the image obtainer, a series being a sequence of medical images as one unit.

8. The image display apparatus according to claim 1, wherein
the target region is a human lung field including a diaphragm, and
the hardware processor generates the display image based on, among the medical images obtained by the image obtainer, medical images corresponding to at least one human respiration period including an inhalation period and an exhalation period as one unit.

9. The image display apparatus according to claim 1, wherein the hardware processor
determines whether or not the medical images obtained by the image obtainer include medical images of a dynamic image of the target region that has cyclical movement, the dynamic image being taken through serial radiography along the time axis, and
generates the display image based on the medical images that are included in the medical images obtained by the image obtainer and are determined to be the dynamic image.

10. The image display apparatus according to claim 1, wherein the hardware processor generates the display image based on processed medical images that are the medical images obtained by the image obtainer and processed.

11. The image display apparatus according to claim 3, wherein the hardware processor generates the display image based on, among the medical images obtained by the image obtainer, a series being a sequence of medical images as one unit.

12. The image display apparatus according to claim 3, wherein
the target region is a human lung field including a diaphragm, and
the hardware processor generates the display image based on, among the medical images obtained by the image obtainer, medical images corresponding to at least one human respiration period including an inhalation period and an exhalation period as one unit.

13. The image display apparatus according to claim 3, wherein the hardware processor
determines whether or not the medical images obtained by the image obtainer include medical images of a dynamic image of the target region that has cyclical movement, the dynamic image being taken through serial radiography along the time axis, and
generates the display image based on the medical images that are included in the medical images obtained by the image obtainer and are determined to be the dynamic image.

14. The image display apparatus according to claim 3, wherein the hardware processor generates the display image based on processed medical images that are the medical images obtained by the image obtainer and processed.

15. An image display method comprising:
obtaining a plurality of medical images of a same target region taken through serial radiography along a time axis;
generating a display image based on the obtained medical images; and
displaying the generated display image, wherein
in generating the display image, the medical images are combined into one composite medical image as the display image.

16. An image display method comprising:
obtaining a plurality of medical images of a same target region taken through serial radiography along a time axis;
extracting feature points from the respective obtained medical images;
laying the extracted feature points on one selected medical image that is selected from among the medical images, thereby generating a display image; and
displaying the generated display image.

17. A non-transitory computer-readable storage medium storing an image display program to cause a computer of an image display apparatus to:
obtain a plurality of medical images of a same target region taken through serial radiography along a time axis;
generate a display image based on the obtained medical images; and
display the generated display image, wherein
the display image is one composite medical image into which the medical images are combined.

18. A non-transitory computer-readable storage medium storing an image display program to cause a computer of an image display apparatus to:
obtain a plurality of medical images of a same target region taken through serial radiography along a time axis;
extract feature points from the respective obtained medical images;
lay the extracted feature points on one selected medical image that is selected from among the medical images, thereby generating a display image; and
display the generated display image.
